(19) United States
(12) Patent Application Publication
(10) Pub. No.: US 2023/0397925 A1
(43) Pub. Date: Dec. 14, 2023

(54) BALLOON CATHETER WITH SELECTIVE SCORING CAPABILITY

(71) Applicant: BARD PERIPHERAL VASCULAR, INC., Franklin Lakes, NJ (US)

(72) Inventor: Matt Casiraro, Tempe, AZ (US)

(21) Appl. No.: 18/034,572

(22) PCT Filed: Nov. 6, 2020
(86) PCT No.: PCT/US2020/059353

§ 371 Date: Apr. 28, 2023

(51) Int. Cl. A61B 17/3207

(52) U.S. Cl. CPC A61B 17/320725; A61B 2017/320733; A61M 25/0082

(57) ABSTRACT

A catheter (10) including a shaft (14), a balloon (12) positioned at the distal end of the shaft, and at least one scoring wire (30) to score a vascular lesion. A distal end portion of the scoring wire is retractable and includes an atraumatic tip (32). The device includes an actuator (40), such as a thumb wheel (44), for advancing the at least one scoring wire to a location such that a portion thereof is alongside the balloon, thereby allowing for selective use in a scoring configuration. When the balloon expands when fluid is delivered to the balloon through the inflation lumen, this expansion pushes the scoring wire against the vascular lesion.

### BACKGROUND OF THE INVENTION

Balloon dilatation catheters are used to treat lesions in vessels. However, difficulties are encountered in navigating tortuous anatomy and safely crossing very tight lesions. Moreover, some lesions are difficult to dilate using just a balloon, and require a focused force to dilate the lesion at safe inflation pressures.

U.S. Pat. No. 6,394,995 to Solar et al. describes a system used to provide enhanced force to treat a lesion. This system has a flexible advancement member with a tracking member slidable over a guidewire, and a balloon having a distal end attached to the tracking member. This type of system fixes the location of the scoring wires, which can limit flexibility in certain applications.

U.S. Pat. No. 7,708,753 to Hardert discloses a scoring balloon catheter that in one embodiment includes translating scoring wires with distal tips. However, these scoring wires lack atraumatic tips. Consequently, the tips of the scoring wires may catch on and potentially perforate the balloon during the advance, which may be deleterious and otherwise make the scoring wires difficult to deploy.

Accordingly, a need is identified for a balloon catheter that can be used either as a conventional, non-scoring balloon catheter or for providing scoring functionality, when such is desired. Deployment of the scoring wire(s) would be achieved in an easy and expedient manner, without a significant risk of hang-up or perforation. Additionally, or alternatively, the scoring wire(s) may also be actuated in a manner that does not require modification to the catheter shaft.

### SUMMARY OF THE INVENTION

An object of the disclosure is a balloon catheter that can be used either as a conventional, non-scoring balloon catheter or for providing scoring functionality, when such is desired.

In one aspect, the disclosure relates to a scoring balloon catheter. The scoring balloon catheter includes a shaft having a proximal end section and a distal end section. An inflatable balloon is mounted on the distal end section of the shaft. At least one scoring wire is partially located within the shaft proximal of the inflatable balloon and including an atraumatic tip adapted for translating in a longitudinal direction to position a portion of the at least one scoring wire alongside the balloon. The atraumatic tip is located proximal of the balloon in a home condition.

In one embodiment, the catheter includes a plurality of scoring wires. Each of the plurality of scoring wires may include an atraumatic tip adapted for translating in a longitudinal direction. In some examples, the atraumatic tip comprises a generally spherical bead forming an end of the scoring wire.

In one embodiment, an actuator is provided for advancing and retracting the at least one scoring wire. The actuator may be connected to the proximal end section of the shaft, such as to a hub associated therewith. The actuator may comprise a thumb wheel rotatably mounted to the hub, or instead a slide supported or carried by the shaft for longitudinal movement to and fro to cause actuation or retraction, respectively.

According to another aspect of the disclosure, a scoring balloon catheter includes a shaft having a proximal end section and a distal end section and a hub connected to the proximal end section of the shaft. An inflatable balloon is mounted on the distal end section of the shaft. An actuator supported by the hub is provided for advancing and retracting at least one scoring wire in a longitudinal direction such that a portion thereof extends alongside the inflatable balloon.

In one embodiment, the at least one scoring wire includes an atraumatic tip adapted for translating in a longitudinal direction. The catheter may include a plurality of scoring wires. Each of the plurality of scoring wires may include an atraumatic tip adapted for translating in a longitudinal direction.

The actuator may be adapted for advancing and retracting the plurality of scoring wires in concert. For example, the actuator may comprise a thumb wheel or a slide. The actuator may be rotatably mounted to the hub, or slidably mounted to the shaft.

Still a further aspect of the disclosure pertains to a scoring balloon catheter. The catheter includes a shaft having a proximal end section and a distal end section. An inflatable balloon is mounted on the distal end section of the shaft. A rotary actuator is provided for advancing and retracting at least one scoring wire in a longitudinal direction such that a portion of the at least one scoring wire extends alongside the inflatable balloon.

In one embodiment, a hub is connected to the proximal end section of the shaft. In such case, the rotary actuator is supported by the hub. The rotary actuator may comprise a thumb wheel rotatably mounted to the hub.

In one embodiment, the at least one scoring wire comprises an atraumatic tip. For example, the atraumatic tip may comprise a generally spherical bead. The catheter may also include a plurality of scoring wires, and the rotary actuator may be adapted for advancing the plurality of scoring wires, and also retracting the same.

### BRIEF DESCRIPTION OF THE DRAWINGS

The above and further advantages of the present invention may be better understood by referring to the following description in conjunction with the accompanying drawings in which:

FIG. 1 is a perspective view of a balloon catheter.

FIG. 2 is a cutaway rear perspective view of the balloon portion of the balloon catheter of FIG. 1.

FIG. 2A is a cross-sectional view taken along line 2A-2A of FIG. 2.

FIG. 3 illustrates a balloon catheter according to one embodiment of the disclosure.

FIG. 3A is a cross-sectional view taken along line 3A-3A of FIG. 3.

FIG. 4 illustrates the balloon catheter of FIG. 3 in an alternative configuration.

FIG. 5 is a cutaway side schematic view of an actuator for use with one or more scoring wires of a balloon catheter.

FIG. 5A is a cross-sectional end view of the actuator taken along line 5A-5A of FIG. 5.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth to provide a thorough understanding of the present invention. The disclosed embodiments may be practiced without these specific details. In other instances, well-known methods, procedures, components, or structures may not have been described in detail so as not to obscure the present inventive concepts.

The invention is not limited in its application to the details of construction and the arrangement of the components set forth in the following description or illustrated in the drawings. The inventive concepts disclosed are capable of other embodiments or of being practiced or carried out in various ways. Also, it is to be understood that the phraseology and terminology employed herein are for the purpose of description and should not be regarded as limiting.

Certain features of the disclosed embodiments that are, for clarity, described in the context of separate embodiments, may also be provided in combination in a single embodiment. Conversely, various features of the invention that are, for brevity, described in the context of a single embodiment, may also be provided separately or in any suitable sub-combination.

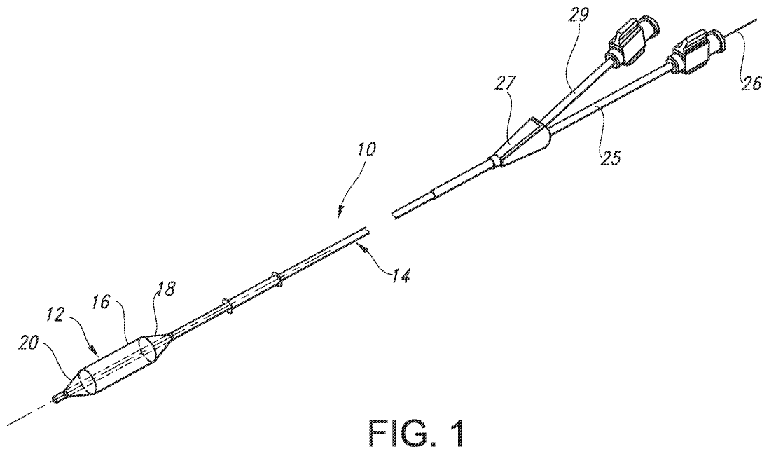

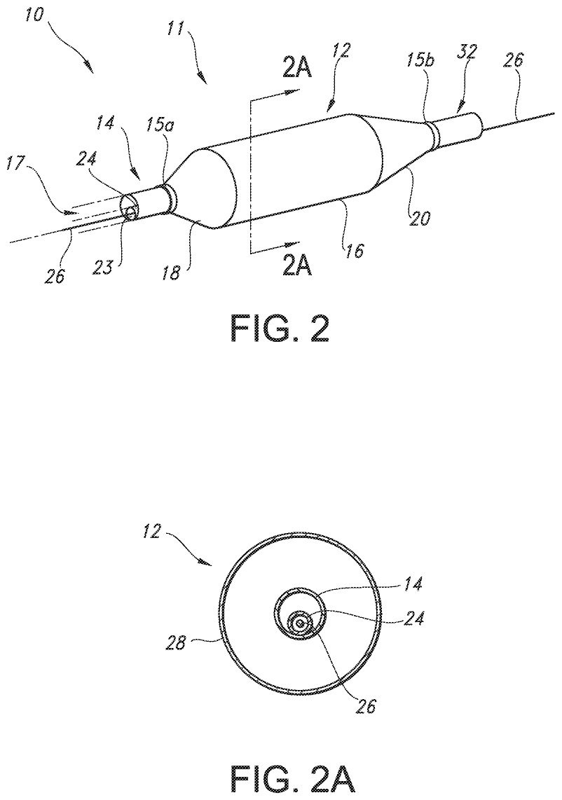

With reference to FIGS. 1, 2, and 2A, provided is a catheter 10 having a distal end portion 11 with a balloon 12 mounted on a catheter shaft 14. The balloon 12 has an intermediate portion 16, or “barrel,” and end portions 18, 20. In one embodiment, the end portions 18, 20 reduce in diameter to join the intermediate portion 16 to the catheter shaft 14 (and thus portions 18, 20 are generally termed cones or cone portions). The balloon 12 is sealed at balloon ends (proximal end 15a and distal end 15b) on the cone portions 18, 20 to allow the inflation of the balloon 12 via one or more inflation lumens 17 extending within catheter shaft 14 and communicating with the interior of the balloon 12.

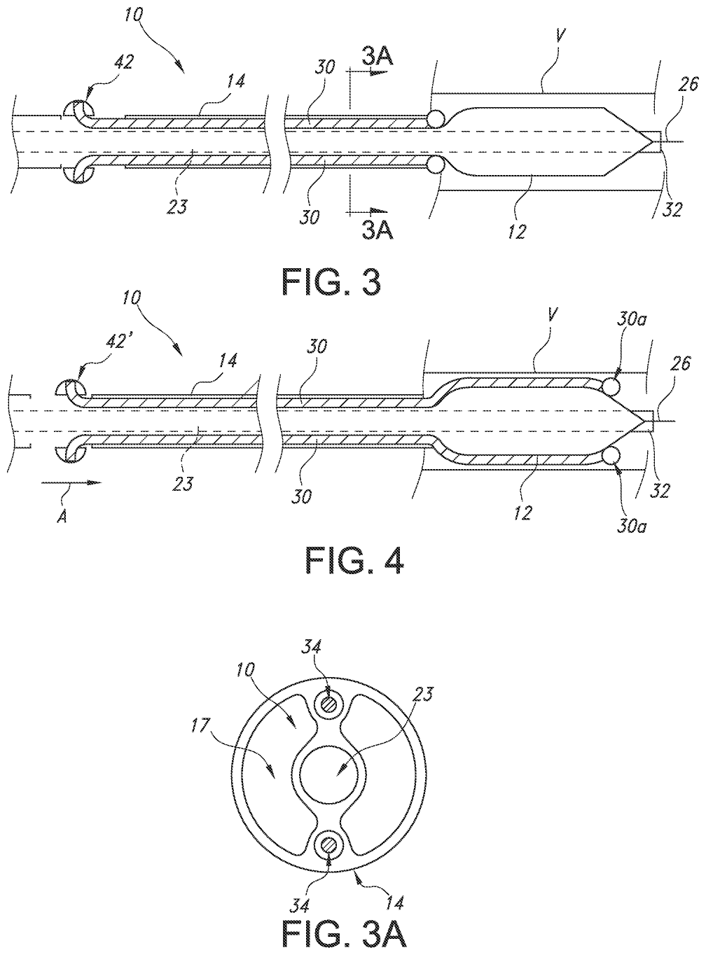

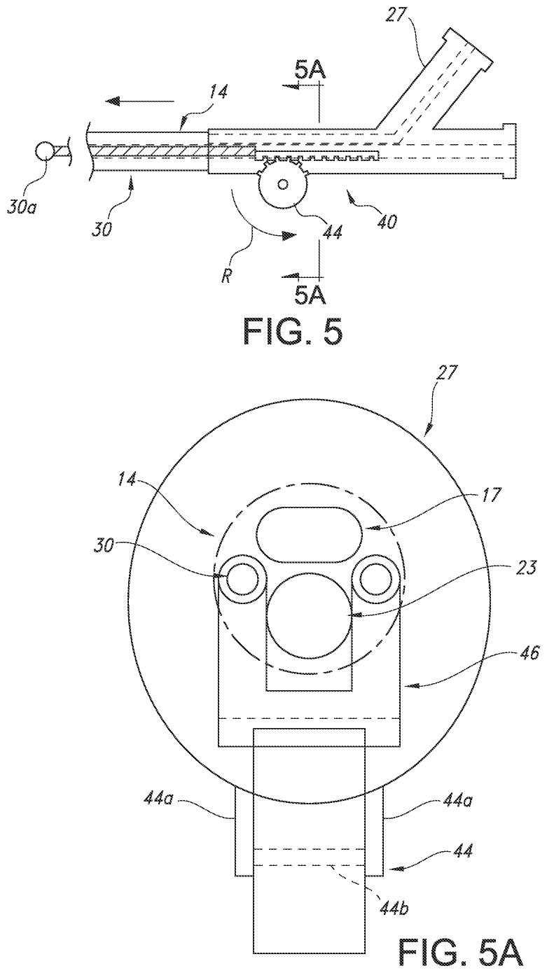

The catheter shaft 14 also includes an elongated, tubular shaft 24 forming a guidewire lumen 23 that directs the guidewire 26 through the catheter 10, and along the distal end portion of which the balloon 12 may be located. As illustrated in FIG. 2, this guidewire 26 may extend through the proximal end portion of the catheter 10 and a first port 25 of a connector or hub 27 into the lumen 23 to achieve an “over the wire” (OTW) arrangement, but could also be provided in a “rapid exchange” (RX) configuration (in which the guidewire 26 exits a lateral opening closer to the distal end, not shown) or else is fed through the tip at a passage distally of the balloon 12 (“short” RX; not shown). A second port 29 may also be associated with catheter 10, such as by way of connector or hub 27, for introducing a fluid (e.g., saline solution, a contrast agent, or both) into the interior compartment of the balloon 12 via the inflation lumen 17 (which may comprise a single structure in connector or hub 27, as shown in FIG. 5A, but may form multiple chambers in the shaft 14, as shown in FIG. 3A).

Inflatable balloon 12 may include a single or multi-layered balloon wall 28 forming the interior for receiving the inflation fluid. The balloon 12 may be a non-compliant balloon having a balloon wall 28 that maintains its size and shape in one or more directions when the balloon is inflated. The balloon 12 in such case also has a pre-determined surface area that remains constant during and after inflation, also has a pre-determined length and pre-determined diameter that each, or together, remain constant during and after inflation. However, the balloon 12 could be semi-compliant or compliant instead, depending on the particular use. The balloon 12 can have any of a variety of diameters ranging from 1.25-40 mm or 2.0-8.0 mm. In any of the embodiments set out herein, inflatable balloon 12 can have any of a variety of lengths such as 10-300 mm or 20-300 mm. Longer balloons may be particularly useful for treating peripheral lesions, which often have long diseased portions.

According to a first aspect of the disclosure, and with reference to FIGS. 3, 3A and 4, the catheter 10 may be provided with one or more scoring wires 30. The pair of scoring wires 30 shown in this example are adapted for being selectively advanced or retracted (compare FIGS. 3 and 4) alongside of the balloon 12, and thus is not connected at a distal end to the catheter 10, such as adjacent to tip 32 distal of the balloon 12. At a distal end, the scoring wire(s) may extend at least partially into the shaft 14 via lumens 34, as shown in FIG. 3A, and may alight therefrom via openings located adjacent to a proximal end 15a of the balloon 12.

In order to advance and retract the scoring wire(s) 30, an actuator 40 may be provided. As shown in FIGS. 3 and 4, this actuator 40 may comprise a slide 42 coaxial with shaft 14 and attached to a proximal end of the scoring wire(s) 30, such as by a bend therein. The slide 42 may be adapted to move to and fro along a proximal portion of the shaft 14 in a generally longitudinal direction. The movement may be from a proximal or retracted position of the slide 42, by which the scoring wire(s) 30 are retracted, to a distal or advanced position (in the direction of arrow A, note forward position of slide 42′), by which the scoring wire(s) are advanced to extend alongside the balloon 12. The advancement would typically be done while the balloon 12 is in a deflated condition, and then the balloon 12 expanded or inflated with the wire(s) 30 alongside as shown to provide the desired scoring function.

In order to help ensure the smooth advance and retraction, the scoring wire(s) 30 may be provided with an atraumatic tip (that is, the distal-most free end of the wire). In the illustrated embodiment, the atraumatic tip comprises a smooth, rounded tip 30a, such as in the form of a generally spherical bead, attached to or integrally formed with a distal end of the wire(s). This atraumatic tip helps to ensure that the wire(s) 30 do not perforate the balloon 12 when being advanced, and further help to ensure that the wire(s) may be advanced without engaging or becoming snagged on adjacent structures, including the associated vessel. Moreover, the tip 30a may be oversized to prevent the wire 30 from inadvertently entering the distal opening of the associated lumen 34 in shaft 14 adjacent the proximal end 15a of the balloon 12.

As can be appreciated, the ability for selective advancement and retraction allows for the scoring wire 30 to be selectively used in connection with the catheter 10 or not (such as for completing percutaneous transluminal angioplasty (PTA) without a scoring function, in which case a scoring wire would not be deployed). In other words, the catheter 10 may be useful as a standard PTA catheter, or used with one or more scoring wires, as desired to achieve a particular treatment effect.

The actuator 40 may take alternative forms besides the slide 42 shown in FIGS. 3 and 4. For example, as shown in FIGS. 5 and 5A, the actuator 40 may comprise a rotary actuator, such as a thumb wheel 44 rotatably connected to the connector or hub 27 (such as by upstanding mounts 44a and axle 44b, shown in phantom). Rotation of the thumb wheel 44, such as in a counterclockwise direction as indicated by arrow R, may cause a toothed portion of it to engage and advance a rack 46 with mating teeth connected to a proximal end of the scoring wire 30 (or more than one scoring wire, if such is present, in which case rack 46 may be adapted to connect to multiple wires, such as by being yoked or U-shaped as shown in FIG. 5A). The length of the advance of the rack 46 may correspond with the maximum deployment of the distal end portion of the scoring wire to adjacent a tip 32 of the catheter 10, as shown in FIG. 4. When retraction is desired, the thumb wheel 44 is simply rotated in the opposite direction, such as once the balloon 12 is deflated after a scoring procedure is complete.

As can be appreciated, this rotary arrangement for actuation allows for the clinician to comfortably and securely grip the connector or hub 27 in the typical fashion during the course of catheter 10 placement, and when desired, use the thumb wheel 44 to selectively deploy the scoring wire(s) 30 at least partially alongside the balloon 12. By using this rotatable type of actuator 40, modification of the shaft 14 is unnecessary from a conventional scoring wire catheter using scoring wire(s) attached to the balloon tip, which makes this a low cost and readily adaptable solution to providing a retractable configuration. Maintaining a thumb on the wheel 44 during the procedure also allows the clinician to easily dial in the amount of the advance or retraction (which can be seen under fluoroscopy) using only a single hand.

As can be understood from FIGS. 3 and 4, when scoring functionality is desired, the scoring wire(s) 30 (two shown in this figure spaced 180 degrees apart around the balloon 12, but it could be only one scoring wire or more than two scoring wires) may be connected to the catheter 10. The physician then gains access to the vessel V, inserts a guidewire 26 into the vessel V, and advances the guidewire 26 through the patient's vasculature under ultrasound, magnetic resonance, fluoroscopic, or some other type of guidance. Once the physician places the guidewire 26 at a satisfactory site, the physician threads the proximal end of the guidewire 26 into the guidewire lumen 23.

The clinician maneuvers the catheter 10 along the guidewire until inflatable balloon 12 reaches the desired position near the site of a lesion (not shown). Typically, this position will allow at least one scoring wire 30 to effectively engage the lesion when the balloon 12 is inflated. However, as can be appreciated, because the scoring wire 30 is retracted during the advance to the lesion, the portion of the catheter 10 including the balloon 12 is of reduced diameter, which may improve trackability.

Once in the desired position in the vessel V, the scoring wire(s) 30 are deployed or advanced, and the inflatable balloon 12 inflated by a source of pressurized fluid (e.g., saline solution) delivered by the inflation lumen(s) 17. The clinician inflates inflatable balloon 12 until scoring wire(s) 30 firmly press into or crack the lesion. Once lesion treatment with catheter 10 is complete, the physician deflates inflatable balloon 12, which allows scoring wire(s) 30 to relax away from the lesion and from the wall of the vessel V. The scoring wire(s) 30 may then be retracted, and the catheter 10 withdrawn or advanced to another treatment location (with or without the use of scoring wire(s) 30. Alternatively, the balloon 12 may be reinflated to treat the now-scored lesion without the scoring wire(s) in place alongside the balloon.

Scoring wire(s) 30 may contact the lesion as long as inflatable balloon 12 remains inflated. The inflation time corresponds to the times the physician chooses for scoring wire to contact the lesion. Inflation times may range from 5 seconds to 5 minutes. The nature of the lesion may determine the appropriate inflation time and inflation speed.

In some embodiments the diameter of scoring wire 30 (or wires, if present) is between 0.003 inches and 0.040 inches, or 0.005 inches and 0.015 inches, 0.008 inches and 0.012 inches.

In some embodiments, scoring wire 30 is 0.10 inches. Scoring wire 30 need not have a uniform diameter, and may have a larger diameter at a distal end as compared to a proximal end, or visaversa. In some embodiments, scoring wire 30 comprises metal, metal alloy, polymer, and/or a shape memory material.

Summarizing, this disclosure may be considered to relate to the following items:
1. A scoring balloon catheter, comprising:
   a shaft having a proximal end section and a distal end section;
   an inflatable balloon mounted on the distal end section of the shaft; and
   at least one scoring wire partially located within the shaft proximal of the inflatable balloon and including an atraumatic tip adapted for translating in a longitudinal direction to position a portion of the at least one scoring wire alongside the balloon.
2. The scoring balloon catheter of item 1, further including a plurality of scoring wires, each scoring wire including an atraumatic tip adapted for translating in a longitudinal direction.
3. The scoring balloon catheter of item 1 or item 2, further including an actuator for advancing and retracting the scoring wire(s).
4. The scoring balloon catheter of item 3, further including a hub connected to the proximal end section of the shaft, and wherein the actuator is connected to the hub.
5. The scoring balloon catheter of item 4, wherein the actuator comprises a thumb wheel rotatably mounted to the hub.
6. The scoring balloon catheter any of items 1-5, wherein the atraumatic tip comprises a generally spherical bead.
7. The scoring balloon catheter of any of items 1-6, wherein the atraumatic tip of the at least one scoring wire is located proximal of the balloon in a home position.
8. A scoring balloon catheter, comprising:
   a shaft having a proximal end section and a distal end section;
   a hub connected to the proximal end section of the shaft;
   an inflatable balloon mounted on the distal end section of the shaft; and
   at least one scoring wire;
   an actuator supported by the hub for advancing and retracting the at least one scoring wire in a longitudinal direction such that a portion thereof extends alongside the inflatable balloon loon.
9. The scoring balloon catheter of item 8, wherein the at least one scoring wire includes an atraumatic tip adapted for translating in a longitudinal direction.
10. The scoring balloon catheter of item 8 or item 9, further including a plurality of scoring wires.
11. The scoring balloon catheter of item 10, each of the plurality of scoring wires includes an atraumatic tip adapted for translating in a longitudinal direction.
12. The scoring balloon catheter of item 10 or item 11, wherein the actuator is adapted for advancing the plurality of scoring wires.
13. The scoring balloon catheter of any of items 8-12, wherein the actuator comprises a thumb wheel rotatably mounted to the hub.
14. A scoring balloon catheter, comprising:
   a shaft having a proximal end section and a distal end section;
   an inflatable balloon mounted on the distal end section of the shaft;
   at least one scoring wire; and
   a rotary actuator for advancing and retracting the at least one scoring wire in a longitudinal direction such that a portion of the at least one scoring wire extends alongside the inflatable balloon.
15. The scoring balloon catheter of item 14, further including a hub connected to the proximal end section of the shaft, and wherein the rotary actuator is supported by the hub.
16. The scoring balloon catheter of item 14 or item 15, wherein the rotary actuator comprises a thumb wheel rotatably mounted to the hub.
17. The scoring balloon catheter of any of items 14-16, wherein the at least one scoring wire comprises an atraumatic tip.
18. The scoring balloon catheter of item 17, wherein the atraumatic tip comprises a generally spherical bead.
19. The scoring balloon catheter of any of items 14-18, further including a plurality of scoring wires.
20. The scoring balloon catheter of item 19, wherein the rotary actuator is adapted for advancing the plurality of scoring wires.

Although the invention has been described in conjunction with specific embodiments, many alternatives, modifications, and variations will be apparent to those skilled in the art. Accordingly, it embraces all such alternatives, modifications, and variations that fall within the appended claims' spirit and scope. It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment may also be illustratively described and presented in the context or format of a plurality of separate embodiments.

All publications, patents and patent applications mentioned in this specification are herein incorporated in their entirety by reference into the specification, to the same extent as if each individual publication, patent or patent application was specifically and individually indicated to be incorporated herein by reference. In addition, or identification of any reference in this application shall not be construed as an admission that such reference is available as prior art to the present disclosure.

1. A scoring balloon catheter, comprising:
a shaft having a proximal end section and a distal end section;
an inflatable balloon mounted on the distal end section of the shaft; and
at least one scoring wire partially located within the shaft proximal of the inflatable balloon and including an atraumatic tip adapted for translating in a longitudinal direction to position a portion of the at least one scoring wire alongside the balloon.

2. The scoring balloon catheter of claim 1, further including a plurality of scoring wires, each scoring wire including an atraumatic tip adapted for translating in a longitudinal direction.

3. The scoring balloon catheter of claim 1, further including an actuator for advancing and retracting the at least one scoring wire.

4. The scoring balloon catheter of claim 1, further including a hub connected to the proximal end section of the shaft, and wherein the actuator is connected to the hub.

5. The scoring balloon catheter of claim 4, wherein the actuator comprises a thumb wheel rotatably mounted to the hub.

6. The scoring balloon catheter of claim 1, wherein the atraumatic tip comprises a generally spherical bead.

7. The scoring balloon catheter of claim 1, wherein the atraumatic tip of the at least one scoring wire is located proximal of the balloon in a home position.

8. A scoring balloon catheter, comprising:
a shaft having a proximal end section and a distal end section;
a hub connected to the proximal end section of the shaft;
an inflatable balloon mounted on the distal end section of the shaft; and
at least one scoring wire;
an actuator supported by the hub for advancing and retracting the at least one scoring wire in a longitudinal direction such that a portion thereof extends alongside the inflatable balloon.

9. The scoring balloon catheter of claim 8, wherein the at least one scoring wire includes an atraumatic tip adapted for translating in a longitudinal direction.

10. The scoring balloon catheter of claim 8, further including a plurality of scoring wires.

11. The scoring balloon catheter of claim 10, each of the scoring wires including an atraumatic tip adapted for translating in a longitudinal direction.

12. The scoring balloon catheter of claim 10, wherein the actuator is adapted for simultaneously advancing and retracting the plurality of scoring wires.

13. The scoring balloon catheter of claim 8, wherein the actuator comprises a thumb wheel rotatably mounted to the hub.

14. A scoring balloon catheter, comprising:
a shaft having a proximal end section and a distal end section;
an inflatable balloon mounted on the distal end section of the shaft;
at least one scoring wire; and
a rotary actuator for advancing and retracting the at least one scoring wire in a longitudinal direction such that a portion of the at least one scoring wire extends alongside the inflatable balloon.

15. The scoring balloon catheter of claim 14, further including a hub connected to the proximal end section of the shaft, and wherein the rotary actuator is supported by the hub.

16. The scoring balloon catheter of claim 14, wherein the rotary actuator comprises a thumb wheel rotatably mounted to the hub.

17. The scoring balloon catheter of claim 14, wherein the at least one scoring wire comprises an atraumatic tip.

18. The scoring balloon catheter of claim 17, wherein the atraumatic tip comprises a generally spherical bead.

19. The scoring balloon catheter of claim 14, further including a plurality of scoring wires.

20. The scoring balloon catheter of claim 19, wherein the rotary actuator is adapted for simultaneously advancing and retracting the plurality of scoring wires.
